(19) United States
(12) Patent Application Publication
(10) Pub. No.: US 2026/0234102 A1
(43) Pub. Date: Aug. 13, 2026

(54) PRODUCTION OF SUSTAINABLE SULFONATES THROUGH TREATMENT OF RE-REFINED BASE OILS

(71) Applicant: HollyFrontier LSP Brand Strategies LLC, Dallas, TX (US)

(72) Inventors: John Pudelski, Dallas, TX (US); Sander Engelsma, Dallas, TX (US); Rumeysa Caliskan, Dallas, TX (US); Arad Seyed Mehdi, Dallas, TX (US)

(73) Assignee: HollyFrontier LSP Brand Strategies LLC, Dallas, TX (US)

(21) Appl. No.: 19/535,053

(22) Filed: Feb. 10, 2026

Related U.S. Application Data
(60) Provisional application No. 63/757,071, filed on Feb. 11, 2025.

(51) Int. Cl. C07C 303/32; C10G 17/06

(52) U.S. Cl. CPC C07C 303/32; C10G 17/06; C10G 2300/201

(57) ABSTRACT

Sulfonates, including metal sulfonates and over-based sulfonates, can be obtained through treatment of re-refined base oil using a sulfonation agent and an inorganic base. The resulting sulfonates are sustainable with a reduced carbon footprint.

This application claims priority to U.S. Provisional Patent Application Ser. No. 63/757,071, entitled “Production of Sustainable Sulfonates Through Treatment of Re-Refined Base Oils,” filed Feb. 11, 2025, the entire contents of which are hereby incorporated by reference.

### BACKGROUND

The present disclosure relates to the production of sulfonates with reduced carbon footprints by treatment of re-refined base oils.

A product's carbon footprint is the amount of CO₂ generated in the production of a fixed quantity of that product. The petroleum industry generally has a large carbon footprint due to greenhouse gas emissions released during oil and gas extraction, refining, transport, and combustion. Regulators and consumers are currently focused on methods to generate petroleum products that involve lower carbon footprints and are more environmentally friendly and sustainable.

Re-refined base oils are produced through a process that takes used motor oil and refines it to remove contaminants and restore its original properties. Because of this manufacturing method, re-refined base oils have product carbon footprints that are 40-80% improved vs. producing virgin base oils. Because of their improved carbon footprints, re-refined base oils are increasingly used in products and applications where there is a desire to demonstrate improved sustainability and are increasingly sought by customers who value this sustainability.

Notably, any additional substances or products produced through or obtained from further processing or treatment of re-refined base oils will also be considered re-refined, sustainable products. Products that would normally produce a large amount of carbon dioxide or require a great deal of energy during their manufacture that can, instead, be obtained from re-refined base oils would be highly valued due to their reduced carbon footprint.

### SUMMARY

The present disclosure pertains to the sustainable production of sulfonates, and particularly to methods for producing metal sulfonates and over-based sulfonates by treating re-refined base oils. The sulfonates produced by the present methods have significantly reduced carbon footprints.

Despite stringent refining, re-refined base oils often contain notable amounts of mineral oil aromatic hydrocarbons (MOAHs). Described herein is the use of SO₃ treatment as a simple and effective method for converting the MOAHs contained within some re-refined base oils to sulfonic acids. The sulfonic acids, in turn, can be neutralized by an appropriate inorganic base to form metal sulfonates. The metal sulfonates are valuable specialty chemicals on their own, but now have the added benefit of an improved carbon footprint due to being derived from re-refined base oils. Moreover, the SO₃ treatment and neutralization steps can typically be practiced in current manufacturing equipment. New or different equipment is not expected to be required for handling re-refined base oils. Finally, the metal sulfonates can be further reacted to produce over-based metal sulfonates via processes well known in the industry. In this case however, the over-based metal sulfonates will have improved carbon footprints due to being derived from re-refined base oils.

### BRIEF DESCRIPTION OF THE DRAWINGS

FIG. 1 shows an overview of steps in an exemplary process for obtaining sulfonates, according to preferred embodiments described herein.

FIG. 2 shows Fourier Transform Infrared Spectroscopy (FTIR) of a sodium petroleum sulfonate reference prepared from standard base oil and a sodium petroleum sulfonate produced from re-refined base oil in accordance with preferred embodiments described herein.

FIG. 3 shows FTIR of a sodium sulfonate reference (Na 458) and a sodium sulfonate produced from a re-refined base oil (SN150) in accordance with preferred embodiments described herein.

FIG. 4 shows FTIR of a sodium sulfonate reference (Na 458) and a sodium sulfonate produced from a re-refined base oil (SN300) in accordance with preferred embodiments described herein.

FIG. 5 shows FTIR of a sodium sulfonate reference (Na 458) and a sodium sulfonate produced from a re-refined base oil (SN500P) in accordance with preferred embodiments described herein.

FIG. 6 shows FTIR of a sodium sulfonate reference (Na 425) and a sodium sulfonate produced from a re-refined base oil (IT80R) in accordance with preferred embodiments described herein.

FIG. 7 shows FTIR of a sodium sulfonate reference (Na 425) and a sodium sulfonate produced from a re-refined base oil (IT80B) in accordance with preferred embodiments described herein.

### DETAILED DESCRIPTION OF PREFERRED EMBODIMENTS

The present disclosure relates to production of sulfonates through treatment of re-refined base oils. The methods disclosed herein produce mineral oil-based sulfonates with carbon footprint advantages from re-refined base oil.

In preferred embodiments, the methods disclosed herein utilize re-refined base oils. As used herein, the term “re-refined base oil” means a base oil made from used motor oil that has been treated or refurbished to remove contaminants. A re-refined base oil has already been subjected to one or more refinement processes, such as dehydration, de-fueling, distillation, hydrotreating and solvent extraction/dewaxing and is considered a sustainable, reusable resource. The treatment steps used to restore the used oil to a higher quality base oil produce less carbon dioxide and thus provide a smaller carbon footprint than the higher quality base oil would have if it was obtained from an original source. Preferred embodiments utilize re-refined base oils with relatively higher amounts of aromatic content remaining, but without significant additives. Examples of useful starting materials include G1 (Group I) re-refined base oils. A G1 base oil typically has less than 90 percent saturates, has been only solvent-refined, and has a higher aromatic content (greater than 1-3%). If the re-refined base oil has too many residual additives it is expected that these would interfere with the sulfonation process or be detrimental to the product performance. On the other hand, if the re-refined base oil has been refined to a high degree, there won't be enough mineral oil aromatic hydrocarbons (MOAH) to sulfonate and thus produce high yields of metal sulfonates. G2 (Group II) and G3 (Group III) re-refined base oils will have lower MOAH content and will give lower yields of sulfonate, but these oils may be used in preferred embodiments of the methods described herein as well. Re-refined base oils are available commercially from various suppliers.

Preferred embodiments of the methods described herein involve sulfonation of the re-refined base oil using a sulfonation agent. A variety of sulfonation agents could be used, including SO₃ gas, oleum, sulfuric acid, chloro sulfonic acid, or acetyl sulfate. Oleum is a liquid made up of concentrated sulfuric acid and sulfur trioxide having the formula H₂S₂O₇. Standard sulfonation conditions can be used, including any sulfonating methods used for standard (not re-refined) base oils. In preferred embodiments, SO₃ gas is used such that the weight percent of SO₃ relative to the weight of the re-refined base oil being treated is about 3-5 w %. Reaction progress is preferably monitored by following the exotherm and allowing the reaction to proceed to completion. After the reaction is completed, the reaction mixture is preferably centrifuged to separate an acid tar layer from an acid oil layer. The acid oil layer is retained for further processing.

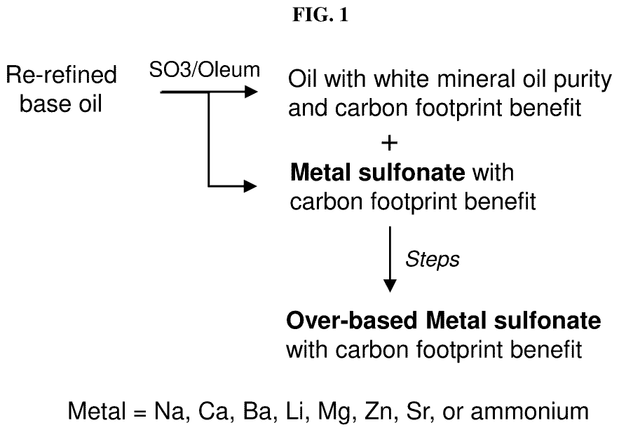

FIG. 1 shows an overview of an exemplary treatment process using re-refined base oil instead of virgin base oil to produce representative metal or over-based sulfonates, all of which have a reduced carbon footprint.

In preferred embodiments, the acid oil layer obtained following the sulfonation is neutralized using an inorganic base, or an inorganic base is otherwise added after or in connection with neutralization. The inorganic base may be a soda solution containing sodium (such as Na₂CO₃), or a slurry of isopropyl alcohol, calcium hydroxide, and water. An inorganic base can be chosen based on the desired metal sulfonate to be obtained and can include sodium, calcium, barium, lithium, magnesium, zinc or strontium inorganic bases. Ammonium sulfonates can be obtained as a non-metal containing variant by using an ammonium base. Additional amounts of isopropyl alcohol, water, and MeOH may be added to assist in neutralization and facilitate separation of layers. A sulfonate layer can be separated, and the resulting sulfonates can be isolated as the metal or inorganic, mineral oil-based sulfonate product. The sulfonate layer can also be allowed to dry to produce a sulfonate product.

In certain preferred embodiment, the metal or inorganic sulfonate product can be further reacted according to known processes to obtain over-based metal sulfonates, also having reduced carbon footprints. Any suitable process and suitable reagents used in the preparation of over-based metal sulfonates from virgin base oils or other suitable starting materials can be used in connection with preferred embodiments of the present methods that use re-refined base oils. For example, a sulfonic acid mixture obtained following sulfonation of the re-refined base oil, or a salt thereof, may be treated with an excess of calcium hydroxide or calcium oxide. Promoters, such as alcohols (methanol, propanol, and the like), glycol ethers, or aromatic hydrocarbons, or combinations thereof, may be introduced to assist in the efficient dispersion of calcium carbonate. Then, carbon dioxide gas may be bubbled through the mixture under controlled temperature and pressure conditions to form the stabilize calcium carbonate micelles required for the final over-based product.

The steps in preferred embodiments of the methods described herein are preferably performed at atmospheric pressure and may be performed at any suitable temperature typically used for these types of treatment steps. Lower or higher temperatures are expected to work, although potentially ineffectively. Low temperatures reduce reaction rates and mixture viscosity which can complicate the process, especially on industrial scale. High temperatures on the other hand can cause over oxidation (carbonization) or a runaway reaction, since the sulfonation reaction is exothermic.

Accordingly, preferred embodiments described herein include a method for producing sulfonates having a reduced carbon footprint using a re-refined base oil. Preferred embodiments of the method include a step of combining a re-refined base oil with a sulfonation agent to produce a sulfonated reaction mixture. The re-refined base oil preferably includes less than 90% saturates and greater than 1% aromatic content. The sulfonation agent preferably includes SO₃, and the sulfonation agent is preferably combined with the re-refined base oil in an amount that provides SO₃ at about 3 to 5 weight percent of the re-refined base oil. An oil layer is preferably obtained from the sulfonated reaction mixture, then the oil layer is neutralized, and an inorganic base is added to the oil layer to produce a neutralized oil layer. A sulfonate layer is preferably separated from the neutralized oil layer, and sulfonates are isolated from the sulfonate layer to produce sulfonates having a reduced carbon footprint. In additional preferred embodiments, the sulfonation agent is SO₃ gas, oleum, sulfuric acid, chloro sulfonic acid, acetyl sulfate, or combinations thereof. In additional preferred embodiments, the inorganic base includes sodium, calcium, barium, lithium, magnesium, zinc, strontium, or ammonium. Further preferred embodiments include a step of reacting the sulfonates from the sulfonate layer with reagents to obtain over-based sulfonates having a reduced carbon footprint. The reagents may be any reagents known to be used for the preparation of over-based sulfonates. Preferably, the sulfonates from the sulfonate layer are reacted with excess calcium hydroxide or calcium oxide, carbon dioxide, and customary and promotive reagents such as alcohols and solvents, to obtain the over-based sulfonates having a reduced carbon footprint.

Additional preferred embodiments described herein include a composition comprising sulfonates having a reduced carbon footprint, wherein the sulfonates are derived from re-refined base oil. Preferably, the sulfonates are prepared according to preferred embodiments of the methods described herein, using re-refined base oils. The sulfonates may be metal sulfonates or over-based sulfonates, according to preferred embodiments described herein.

### Example 1

Exemplary Sodium Sulfonate from Re-Refined Base Oil

Kleen+ RHT 70 (Safety-Kleen, Norwell, MA) (970 g) was sulfonated with 4.5 w % SO₃ gas (43.7 g). Reaction progression was monitored by following the exotherm, indicating completion after 60 minutes. The acid oil was transferred and centrifuged for 8 minutes, resulting in separation between the acid tar and acid oil. A soda solution (16 m %) was added to the acid oil under stirring (300 rpm) at 65° C., until the solution reached PH 8. Subsequently, 40 m % of an isopropyl alcohol (IPA):H₂O (55:45) solution was added. The mixture as a whole was transferred to a separation funnel, where two clear layers formed within 5 minutes. The bottom sulfonate layer was isolated, and soda (Na₂CO₃) was added at 65° C. until saturated. A water layer separated from the sulfonate mixture and was subsequently removed. Next, the mixture was heated to 115° C. for 15 minutes to evaporate off the IPA, providing the sulfonate product as an orange tinted viscous liquid (13.6 g). The product obtained was a mixture of hydrocarbons and sodium sulfonates from re-refined origin.

### Example 2

Exemplary Calcium Sulfonate from Re-Refined Base Oil

The sulfonation was carried out as described for Example 1. Neutralization of the acid oil (200 g) was done with a slurry of 30 m % IPA:Ca(OH)₂:H₂O (65:30:5, 60 g) under stirring (300 rpm) at room temperature, followed by an additional 8 m % MeOH (16 g) and 2.5 m % H₂O (5 g). After 30 minutes, the mixture was vacuum filtered over dicalite and transferred to a separation funnel. 10 m % IPA (20 g) was added to facilitate separation. The layers separated over 10 minutes as 65° C. The top layer was isolated and the IPA and MeOH were removed by heating to 75° C. The concentrate was dried by heating to 115° C. for an additional 15 minutes, yielding a calcium sulfonate that hardened over time.

### Example 3

Characterizing Exemplary Sulfonates from Re-Refined Base Oil

The objective of this example was to compare a Re-refined Na 425 to a reference Na 425 to validate that sodium petroleum sulfonates were successfully produced using re-refined base oil. The reference “Na 425” used was a commercial sodium petroleum sulfonate reference, Sonneborn Petronate™ L. The “Re-refined Na 425” was produced from re-refined base oil according to a method described herein.

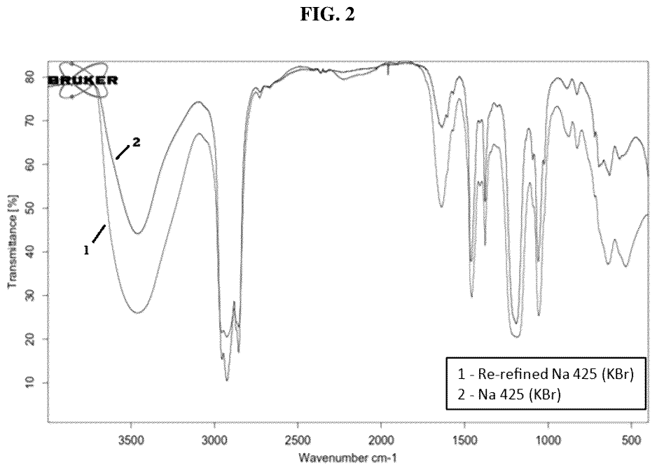

Fourier Transform Infrared Spectroscopy (FTIR), in FIG. 2, showed that the Re-refined Na 425 was similar to Na 425. An FTIR library match confirmed that the Re-refined Na 425 was sodium petroleum sulfonate.

Bruker Q-TOF under negative ESI ionization was used to characterize the petroleum sulfonates. The samples were diluted to ~100 ppm using DCM and MeOH. The scan range was m/z 150-3000. No species with 2 sulfur atoms were detected. Table 1 below lists the composition of Na 425 and Table 2 below lists the composition of Re-refined Na 425.

TABLE 1
Chemical Composition of Na 425

| | | Aromatic |
|---|---|---|
| Chemical Formula | Typical Structures | Type |
| CₙH₂ₙ₋₇O₃S; n = 16-44 | Alkylbenzene | Mono |
| CₙH₂ₙ₋₉O₃S; n = 16-45 | Alkylindane, Alkyltetralin | Mono |
| CₙH₂ₙ₋₁₁O₃S; n = 16-45 | Alkylindene, Alkyldialin | Mono |
| CₙH₂ₙ₋₁₃O₃S; n = 16-45 | Alkylnaphthalene | Di |
| CₙH₂ₙ₋₁₅O₃S; n = 16-44 | Alkylacenaphthene | Di |
| CₙH₂ₙ₋₁₇O₃S; n = 18-44 | Alkylfluorene, | Di |
| | Alkylacenaphthylene | |

TABLE 2
Chemical Composition of Na 425

| | | Aromatic |
|---|---|---|
| Chemical Formula | Typical Structures | Type |
| CₙH₂ₙ₋₁O₃S; n = 16-36 | Cyclohexane | — |
| CₙH₂ₙ₋₃O₃S; n = 19-30 | Decahydronaphthalene | — |
| CₙH₂ₙ₋₅O₃S; n = 17-30 | Tetradecahydroanthracene | — |
| CₙH₂ₙ₋₇O₃S; n = 15-33 | Alkylbenzene | Mono |
| CₙH₂ₙ₋₉O₃S; n = 15-31 | Alkylindane, Alkyltetralin | Mono |
| CₙH₂ₙ₋₁₁O₃S; n = 15-33 | Alkylidene, Alkyldialin | Mono |
| CₙH₂ₙ₋₁₃O₃S; n = 16-29 | Alkylnaphthalene | Di |
| CₙH₂ₙ₋₁₅O₃S; n = 16-22 | Alkylacenaphthene | Di |
| CₙH₂ₙ₋₁₇O₃S; n = 16-22 | Alkylfluorene, | Di |
| | Alkylacenaphthylene | |

The results indicated that Re-refined Na 425 was sodium petroleum sulfonate. However, the composition was not the same compared to Na 425. It contained saturated petroleum sulfonates which were not present in Na 425. The molecular weight range was also lower compared to Na 425. These differences are consistent with the different starting materials used to prepare Re-refined Na 425 and Na 425.

### Example 4

Additional Exemplary Sulfonates from Re-Refined Base Oil

Group 1 re-refined base oils YUNIGREEN SN150, SN300, and SN500P (Yunigreen, Yanbu, Saudi Arabia) were used to obtain sodium sulfonates according to exemplary methods described herein. The physical properties of the re-refined base oils (RRBO's) YUNIGREEN SN150, SN300, and SN500P were measured and are shown below in Table 3.

TABLE 3
Physical Parameters of RRBO's

| Analysis | Unit | SN150 | SN300 | SN500P |
|---|---|---|---|---|
| Viscosity | mm²/s | 31.1 | 54.9 | 63.1 |
| 40° C. | | | | |
| Viscosity | mm²/s | 5.44 | 7.94 | 8.70 |
| 100° C. | | | | |
| Density | kg/m³ | 856.3 | 861.7 | 862.0 |
| 20° C. | | | | |
| Refractive | — | 1.4730 | 1.4755 | 1.4754 |
| Index | | | | |
| <C₂₅% | % | 16.3 | 6.7 | 2.2 |
| UVA | — | 1067 | 905 | 561 |

Treatment of YUNIGREEN SN150. YUNIGREEN SN150 (448 g) was sulfonated with 4.5 w % SO₃ gas, 1.8 eq. Reaction progression was monitored by following the exotherm, indicating completion after 60 minutes. The acid oil was transferred and centrifuged for 8 minutes, resulting in separation between the acid tar (3.3 g, 0.7%) and acid oil. A portion of the acid oil was used for further workup (223 g, 49.7% of total), and the correction factor based on mass balance was used to calculate yields further in the method.

At 65° C. a 40 m % 55:45 IPA:H₂O washing mixture including 15 mL soda (16 w % Na₂CO₃) was used to neutralize the acid oil. Once a pH of 8 was reached the mixture was left to settle at 65° C. for 7 minutes, after which a separation was visible. The layers were separated, and IPA was evaporated from the oil while stirring at 105° C. The bottom layer containing the sulfonates was desalinated to remove excess water. After separation, the solvents were removed by evaporated at 105° C., to provide 8.3 g (16.7 g corr.) sodium sulfonate, corresponding to 95.5% and 3.7% yield respectively. The sulfonate product had a measured sulfonate content of 92%, and blending to the desired active content of 62% resulted in a yield of 24.8 g/5.6%.

To account for material losses during mass transfer a correction factor was applied. The recalculated mass balance is shown below in Table 4.

TABLE 4
Mass balance (re)calculation of SN150 SO₃ treatment

| | |
|---|---|
| Mass Input | Mass Output |

| | | | |
|---|---|---|---|
| Oil | 447.8 | Acid Tar | 3.3 |
| SO₃ | 20.5 | Treated Oil | 468.3 |
| | | Sulfonate | 16.7 |
| Total: | 468.3 | Total: | 446.4 (95.3%) |

Treatment of YUNIGREEN SN300. YuniGreen SN300 (444 g) was sulfonated with 4.5 w % SO₃ gas, 1.8 eq. Reaction progression was monitored by following the exotherm, indicating completion after 60 minutes. The acid oil was transferred and centrifuged for 8 minutes, resulting in separation between the acid tar (20.7 g, 4.9%) and acid oil. A portion of the acid oil was used for further workup (228 g, 54.5% of total), and the correction factor based on mass balance was used to calculate yields further in the method.

At 65° C. a 40 m % 55:45 IPA:H₂O washing mixture including 15 mL soda (16 w % Na₂CO₃) was used to neutralize the acid oil. Once a pH of 8 was reached the mixture was left to settle at 65° C. for 7 minutes, after which a separation was visible. The layers were separated, and IPA was evaporated from the oil while stirring at 105° C. The bottom layer containing the sulfonates was desalinated to remove excess water. After separation, the solvents were removed by evaporated at 105° C., providing 10.7 g (19.6 g corr.) sodium sulfonate, corresponding to 90.5% yield. The sulfonate product had a measured sulfonate content of 74%. Blending to the desired active content of 62% using untreated YuniGreen SN300 resulted in a yield of 23.4 g/5.5%.

To account for material losses during mass transfer a correction factor was applied. The recalculated mass balance is shown below in Table 5.

TABLE 5
Mass balance (re)calculation of SN300 SO₃ treatment

| | |
|---|---|
| Mass Input | Mass Output |

| | | | |
|---|---|---|---|
| Oil | 444 | Acid Tar | 20.7 |
| SO₃ | 19.2 | Treated Oil | 382.4 |
| | | Sulfonate | 19.6 |
| Total: | 463.2 | Total: | 422.7 (91.3%) |

Treatment of YUNIGREEN SN500P. YUNIGREEN SN500P (678 g) was sulfonated with 4.5 w % SO₃ gas, 1.8 eq. Reaction progression was monitored by following the exotherm, indicating completion after 60 minutes. The acid oil was transferred and centrifuged for 8 minutes, resulting in separation between the acid tar (36.4 g, 5.3%) and acid oil.

At 65° C. a 40 m % 55:45 IPA:H₂O washing mixture including 40 mL soda (16 w % Na₂CO₃) was used to neutralize the acid oil. Once a pH of 8 was reached the mixture was left to settle at 65° C. for 9 minutes, after which a separation was visible. The layers were separated, and IPA was evaporated from the oil while stirring at 105° C. The bottom layer containing the sulfonates was desalinated to remove excess water. After separation, the solvents were removed by evaporated at 105° C., providing 45.4 g sodium sulfonate, corresponding to 6.6% yield. The sulfonate product had a measured sulfonate content of 67%, and diluting with RRBO to the desired active content of 62% resulted in an effective yield of 49.1 g/7.2%.

To account for material losses during experimental mass transfer a correction factor was applied. The recalculated mass balance is shown below in Table 6.

TABLE 6
Mass balance (re)calculation of SN500P SO₃ treatment

| | |
|---|---|
| Mass Input | Mass Output |

| | | | |
|---|---|---|---|
| Oil | 678 | Acid Tar | 36.4 |
| SO₃ | 28.5 | Treated Oil | 603.8 |
| | | Sulfonate | 45.4 |
| Total: | 706.5 | Total: | 685.6 (97.0%) |

Summary of results: The SN150 RRBO was successfully treated with SO₃, yielding a sulfonate product at 5.6% (corrected to 62% active concentration) and a MW of approximately 583. The SN300 RRBO was treated with SO₃, yielding a sulfonate product at 5.5% (corrected to 62% active concentration) and a MW of approximately 632. The SN500P grade was treated with SO₃, yielding a sulfonate product at 7.2% (corrected to 62% active concentration) and a MW of approximately 644.

Characterization and Compositional Analysis: Samples of the sulfonate products obtained using each of the RRBO's described above were diluted to 1000 ppm using DCM and then to 100 ppm using MeOH. They were analyzed using the Bruker Q-TOF under negative ESI ionization. The scan range was m/z 150-2000. Results are shown in Table 7 below.

TABLE 7

| Chemical | | | | | Aromatic |
|---|---|---|---|---|---|
| Formula | Na-458 | SN150 | SN300 | SN500 | Type |
| CₙH₂ₙ₋₇O₃S | n = 17-47 | n = 18-47 | n = 18-48 | n = 21-56 | Mono |
| CₙH₂ₙ₋₉O₃S | n = 16-49 | n = 16-49 | n = 18-55 | n = 21-56 | Mono |
| CₙH₂ₙ₋₁₁O₃S | n = 19-48 | n = 17-53 | n = 18-56 | n = 21-57 | Mono |
| CₙH₂ₙ₋₁₃O₃S | n = 20-47 | n = 19-50 | n = 20-57 | n = 24-57 | Di |
| CₙH₂ₙ₋₁₅O₃S | n = 21-46 | n = 19-48 | n = 19-57 | n = 26-57 | Di |
| CₙH₂ₙ₋₁₇O₃S | n = 23-45 | n = 25-48 | n = 19-56 | n = 26-57 | Di |
| CₙH₂ₙ₋₁₉O₃S | — | n = 23-48 | n = 25-57 | n = 27-57 | Poly |
| CₙH₂ₙ₋₂₁O₃S | — | — | n = 50-56 | — | Poly |
| MW: | 487 | 583 | 632 | 644 | |

YUNIGREEN RRBO grades SN150, SN300, and SN500P were successfully sulfonated to produce re-refined RRBO-based sulfonates. All three grades yielded high-concentration sulfonates with a 5-7% conversion rate and achieved approximately 90% UVA reduction after a single treatment.

A UV quantification method was used to determine the aromatic content of the sodium petroleum sulfonates obtained as described above. The method quantifies mono-, di-, and polynuclear aromatic hydrocarbons, and total aromatics, by UV spectrophotometry. A hydrocarbon sample of known concentration is dissolved in a paraffinic solvent and measured in a cell of known path length. Absorbance at 198 nm, 228 nm, and 260 nm is used to determine the respective aromatic classes based on their characteristic UV spectra, which are only minimally affected by alkyl substitution. The method applies to liquid or solid hydrocarbons soluble in paraffinic solvents and includes sulfur-containing aromatic compounds within the corresponding classes. Acenaphthalene, benzothiophene, and dibenzothiophene sulfonates, if present, were included in the diaromatics group, and napthothiophene sulfonates, if present, were included in the polyaromatics group. Na 458 represents a sodium sulfonate reference sample. Results are shown in Table 8 below.

TABLE 8
Percent w/w Aromatics Content

| Aromatics Type | Na 458 | SN150 | SN300 | SN500P |
|---|---|---|---|---|
| Monoaromatics | 55.4823 | 78.7273 | 60.6808 | 56.3082 |
| Diaromatics | 6.0357 | 10.3415 | 9.6223 | 7.9414 |
| Polyaromatics | 0.9818 | 2.9318 | 3.6966 | 2.7497 |
| Total Aromatics | 62.4998 | 92.0006 | 73.9997 | 66.9993 |

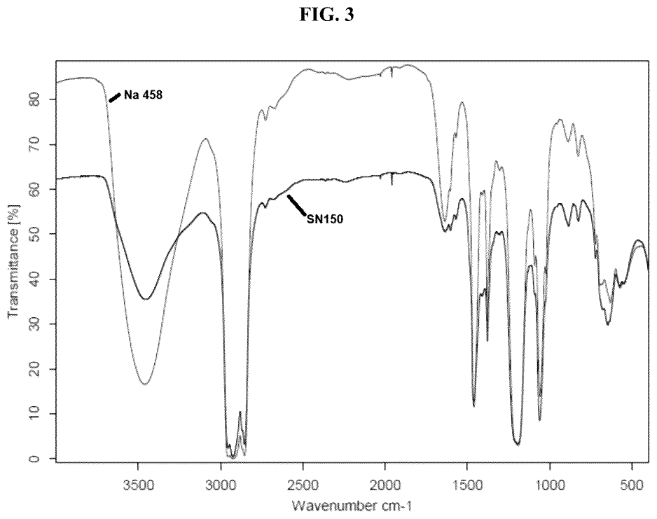

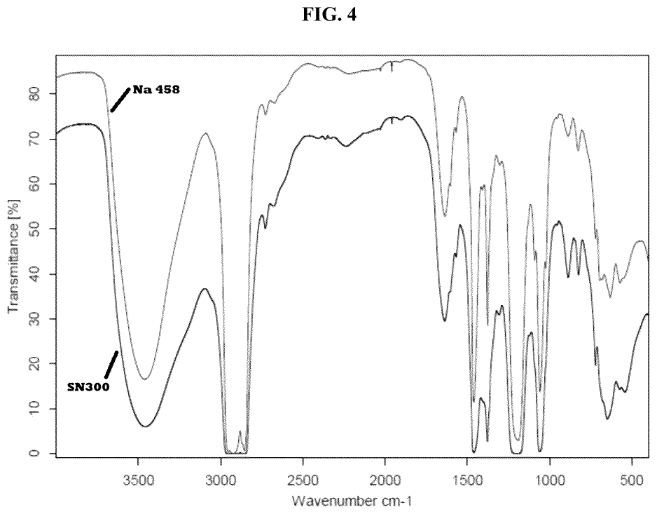

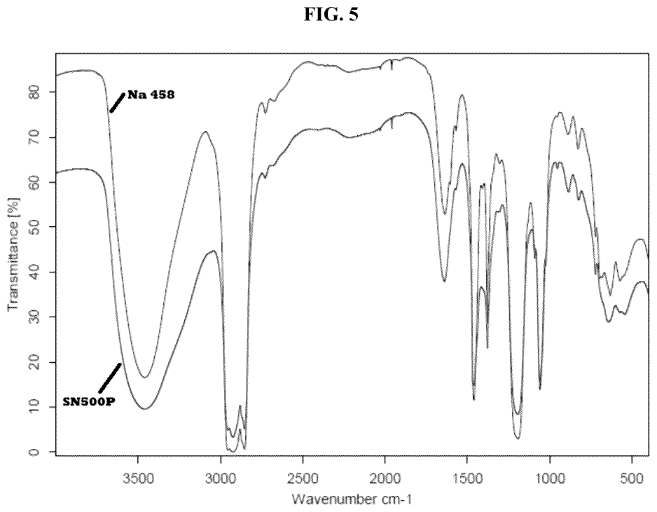

Fourier Transform Infrared Spectroscopy (FTIR) was performed for each sodium petroleum sulfonate and compared to Na 458 as a sodium sulfonate reference. Results are shown in FIG. 3-5.

### Example 5

Additional Exemplary Sulfonates from Re-Refined Base Oil

Group 1 re-refined base oils Itelyum Regeneration HG-3N (IT80R) and Itelyum Lubricant Base oil 80N (IT80B) (Itelyum, Pieve Fissiraga (LO), Italy) were used to obtain sodium sulfonates according to exemplary methods described herein. The physical properties of the re-refined base oils IT80R and IT80B were measured and are shown below in Table 3.

TABLE 9
Physical Parameters of RRBO's

| Analysis | Unit | IT80R | IT80B |
|---|---|---|---|
| Viscosity 40° C. | mm²/s | 15.3 | 14.8 |
| Viscosity 100° C. | mm²/s | 3.54 | 3.48 |
| Density 20° C. | kg/m³ | 842.0 | 842.7 |
| Refractive Index | — | 1.4667 | 1.4678 |
| <C₂₅% | % | 39.3 | 44.2 |
| UVA | — | 782 | 822 |

IT80R and IT80B were reportedly the same oil grade, with IT80R being the more purified variant. Both grades were solvent refined, but IT80R also received hydro-finishing steps. Despite this difference in process no significant difference in UVA was observed, indicating high possible MOAH % present in the RRBO. To test sulfonate production both grades were sulfonated via a SO₃-treatment.

Itelyum IT80R. With a viscosity⁴⁰ of 15.3 cSt IT80R is similar to previously purified RRBO's. Previous SO₃ treatments of high UVA RRBO's did produce sulfonate products at improved yields of 5-7%.

SO₃ treatment of Itelyum IT80R. IT80R (748.4 g) was sulfonated with 4.5 w % SO₃ gas, 1.8 eq. Reaction progression was monitored by following the exotherm, indicating completion after 60 minutes. A large amount of hard tar formed, more akin to a high viscosity feedstock tar. Due to the large density difference between acid oil and tar, layers quickly formed. The acid oil layer was transferred and centrifuged for 8 minutes, resulting in separation between the acid tar (45 g, 6.2%) and acid oil.

At 65° C. a 40 m % 55:45 IPA:H₂O washing mixture including 60 mL soda (16 w % Na₂CO₃) was used to neutralize the acid oil. Once a pH of 8 was reached the mixture was left to settle at 65° C. for 8 minutes, after which a separation was visible. The layers were separated, and IPA was evaporated from the oil while stirring at 105° C. The bottom layer containing the sulfonates was desalinated to remove excess water, and after separation the sulfonates were evaporated at 105° C., to provide 50.2 g sodium sulfonate, corresponding to 87% yield. The sulfonate product had a measured sulfonate content of 75%. Blending to the desired active content of 62% resulted in a yield of 60.7 g/8.3%.

To account for material losses during mass transfer a correction factor was applied. The recalculated mass balance is shown in Table 10.

TABLE 10
Mass balance (re)calculation of IT80R SO₃ treatment

| | |
|---|---|
| Mass Input | Mass Output |

| | | | |
|---|---|---|---|
| Oil | 748 | Acid Tar | 45.0 |
| SO₃ | 36.2 | Treated Oil | 636 |
| | | Sulfonate | 50.2 |
| Total: | 784.2 | Total: | 731.2 (93.2%) |

Itelyum IT80B. Sulfonation of IT80R was performed successfully, with high sulfonate yield.

SO₃ treatment of Itelyum IT80B. Itelyum IT80B (593.2 g) was sulfonated with 4.5 w % SO₃ gas, 1.8 eq. Reaction progression was monitored by following the exotherm, indicating completion after 60 minutes. The acid oil was transferred and centrifuged for 8 minutes, resulting in separation between the acid tar (16.9 g, 3.0%) and acid oil.

At 65° C. a 40 m % 55:45 IPA:H₂O washing mixture including 30 mL soda (16 w % Na₂CO₃) was used to neutralize the acid oil. Once a pH of 8 was reached the mixture was left to settle at 65° C. for 6 minutes, after which a separation was visible. The layers were separated, and IPA was evaporated from the oil while stirring at 105° C. The bottom layer containing the sulfonates was desalinated to remove excess water, and after separation the solvents were removed by evaporated at 105° C., to provide 32.5 sodium sulfonate, corresponding to 91.1% yield. The sulfonate product had a measured sulfonate content of 86%. Blending to the desired active content of 62% resulted in a yield of 45.1 g/8.1%.

To account for losses during mass transfer a correction factor was applied to the measured yields to allow better translation to a plant process. The recalculated mass balance is shown in Table 11.

TABLE 11
Mass balance (re)calculation of IT80B SO₃ treatment

| | |
|---|---|
| Mass Input | Mass Output |

| | | | |
|---|---|---|---|
| Oil | 593.2 | Acid Tar | 16.9 |
| SO₃ | 25.5 | Treated Oil | 507 |
| | | Sulfonate | 32.5 |
| Total: | 618.7 | Total: | 556.4 (89.9%) |

RRBO grades Itelyum Regeneration HG-3N (IT80R) and Itelyum Base oil 80N (IT80B) were successfully sulfonated to produce RRBO-based sodium sulfonates. Both provided high-concentration sulfonates with an 8% conversion rate and achieved approximately 90% UVA reduction after a single SO₃-treatment.

Characterization and Compositional Analysis: Samples of the sulfonate petroleum products obtained using each of the RRBO's described above were diluted to 1000 ppm using DCM and then to 100 ppm using MeOH. They were analyzed using the Bruker Q-TOF under negative ESI ionization. The scan range was m/z 150-2000. Na 425 represents a sodium petroleum sulfonate reference sample. Results are shown in Table 12 below.

TABLE 12

| Chemical | | | | Aromatic |
|---|---|---|---|---|
| Formula | Na 425 | IT80R | IT80B | Type |
| CₙH₂ₙ₋₇O₃S | n = 16-44 | n = 17-43 | n = 17-40 | Mono |
| CₙH₂ₙ₋₉O₃S | n = 16-45 | n = 17-43 | n = 15-41 | Mono |
| CₙH₂ₙ₋₁₁O₃S | n = 16-45 | n = 17-45 | n = 15-41 | Mono |
| CₙH₂ₙ₋₁₃O₃S | n = 16-45 | n = 20-45 | n = 17-41 | Di |
| CₙH₂ₙ₋₁₅O₃S | n = 16-44 | n = 21-44 | n = 19-43 | Di |
| CₙH₂ₙ₋₁₇O₃S | n = 18-44 | n = 21-44 | n = 19-44 | Di |
| CₙH₂ₙ₋₁₉O₃S | — | — | n = 39-43 | Poly |

The UV quantification method described above was used to determine the aromatic content of the sodium petroleum sulfonates obtained in this example. Acenaphthalene, benzothiophene, and dibenzothiophene sulfonates, if present, were included in the diaromatics group, and napthothiophene sulfonates, if present, were included in the polyaromatics group. Na 425 represents a sodium petroleum sulfonate reference sample. Results are shown in Table 13 below.

TABLE 13
Percent w/w Aromatics Content

| Aromatics Type | Na425 | IT80R | IT80B |
|---|---|---|---|
| Monoaromatics | 56.1963 | 67.7139 | 76.7533 |
| Diaromatics | 5.8900 | 6.2662 | 7.6436 |
| Polyaromatics | 9136 | 1.0199 | 1.6031 |
| Total Aromatics | 62.9999 | 75.0000 | 86.0000 |

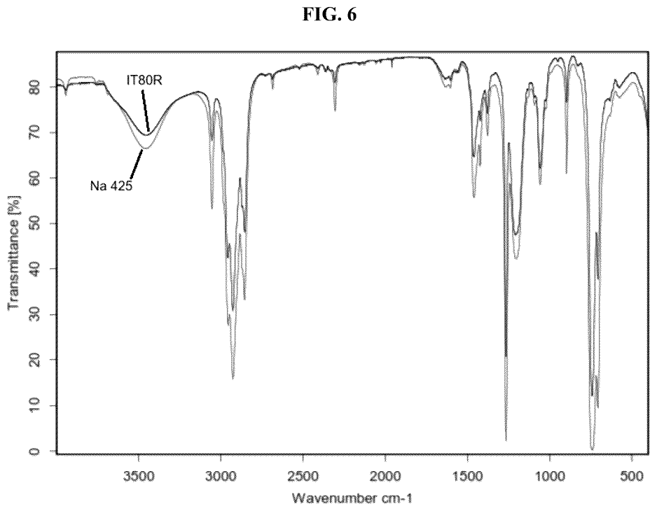

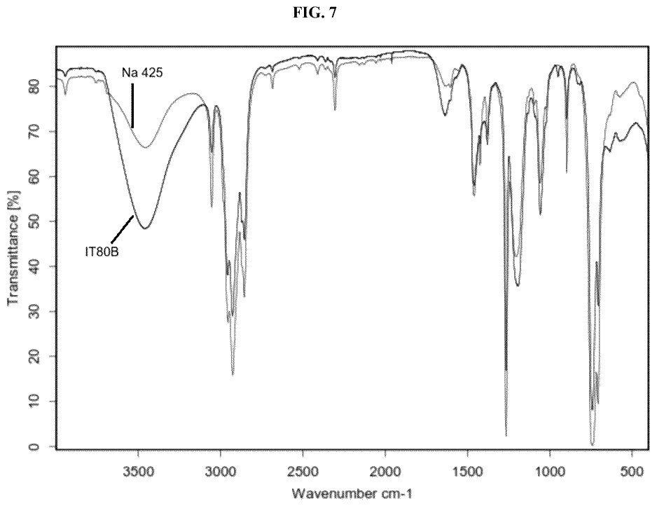

Fourier Transform Infrared Spectroscopy (FTIR) was performed for each sodium petroleum sulfonate and compared to Na 425 as a sodium petroleum sulfonate reference. Results are shown in FIG. 6-7.

### Example 6

Exemplary Calcium Sulfonates from Re-Refined Base Oil

Group 1 re-refined base oil Itelyum Regeneration HG-3N (IT80R) was used to obtain sodium sulfonates according to exemplary methods described herein. This example demonstrates an alternate exemplary method to produce calcium sulfonates of re-refined origin.

SO₃ treatment of Itelyum IT80R. IT80R (454.4 g) was sulfonated with 4.5 w % SO₃ gas, 1.8 eq. Reaction progression was monitored by following the exotherm, indicating completion after 60 minutes. The acid oil was transferred and centrifuged for 8 minutes, resulting in separation between the acid tar and acid oil. 424.7 g acid oil was obtained with a measured SA % of 5.8%. The acid oil was then used in the following steps for conversion to calcium sulfonate.

Calcium neutralization of IT80R acid oil. IT80R acid oil (100 g) was mixed with 19.5 g methanol, 9.0 g Ca(OH)₂, 1.5 g water, corresponding to a 30 m % 65:30:5 MeOH:Ca(OH)₂:H₂O addition. The mixture was stirred at room temperature for 45 minutes using a magnetic stirrer (650 rpm). The mixture was separated via vacuum filtration using a Büchner glass filter funnel covered with dicalite filter earth. The filter cake was washed with 10 g IPA under vacuum. The solvents were removed from the combined liquid fractions by heating to 105° C. under a flow of air for 30 minutes. Once all volatiles were removed the oil/sulfonate mixture (92 g) was cooled down to 65° C. 20 g isopropanol, 17 g heptane, and 5 g deionized water were added and the whole was homogenized under stirring. The wash recipe corresponds to a 45 m % 48:40:12 IPA:Hept:H₂O addition.

The washed mix was placed in a separatory funnel, after which two layers formed. The top layer containing the oil was isolated and volatiles were evaporated at 75° C., yielding a light-yellow colored oil with no odor. The bottom layer containing the sulfonate was heated to 115° C. until it solidified, yielding 5.4 grams of a crystalline product.

Re-refined Calcium Sulfonate Analysis. The attained crystalline product was analyzed to determine its properties, and to prove that it comprised out of calcium sulfonates. Table 12 shows the measured properties, with specifications for a standard commercially produced CaSulf reference shown for comparison.

TABLE 14
Measured IT80R CaSulf Properties

| | | IT80R | CaSulf |
|---|---|---|---|
| Analysis | Unit | CaSulf | Reference |
| TBN | mgKOH/g | <2 | 5-12 |
| Active Sulfonate | % | 79.9 | 43-47 |
| Total Calcium | % | 1.87 (1.05*) | 1.77-2.2 |

*Ca % corrected for a 45% active blend

Hyamine titration showed a content of 80%, agreeing with the formation of sulfonates. ICP measurement showed the presence of calcium ions, proving that the found sulfonates were indeed calcium sulfonates. The product's IR spectrum was measured and compared to a standard CaSulf reference. The IR spectrum showed two main peak ranges. A cluster of sharp peaks at 2900 cm-1 corresponded to the C—H stretch from both the aromatic carbons in the sulfonate group and the aliphatic carbons found in the sulfonate and the oil components of the product. Peaks present between 1000-1250 cm-1 corresponded to S═O and S—O stretch vibrations and indicated the presence of sulfonate/sulfonic acid groups in the sample. The calcium sulfonates attained from IT80R show deeper peaks in this area, corresponding to the relatively high active sulfonate content compared to the reference.

Based on the results of this example, an additional preferred exemplary method for producing calcium sulfonates from re-refined base oils could include the following steps:
1. Weigh acid oil
2. Add 30 m % 65:30:5-MeOH:Ca(OH)₂:H₂O
3. Stir for 45 minutes at room temperature
4. Filter mixture over a Büchner funnel covered with dicalite at 200 mbar pressure
5. Wash filter cake with 10 m % IPA and combine liquid fractions
6. Evaporate all volatile solvents at 105° C.
7. Add 45 m % 48:40:12 IPA:Hept:H₂O relative to the solvent-free oil/sulfonate mixture
8. Set in separation funnel at 65° C. for 10 minutes
9. Separate layers, remove IPA and heptane via evaporation at 75° C.
10. Dry the product at 115° C. until fully solidified
11. Measure sulfonate content, calcium content, IR spectrum, and total base number of product

In summary, IT80R was subjected to SO₃ treatment to produce an acid oil containing 5.8% sulfonic acids. Subsequent neutralization with calcium hydroxide resulted in the formation of a concentrated (80%) calcium sulfonate of re-refined origin, obtained in 5.4 m % yield. When normalized to standard oil content, this corresponds to a yield of 9.6%. These results indicate a high conversion efficiency of sulfonic acids to calcium sulfonates. Compositional analysis of sulfonate content, calcium concentration, and infrared (IR) absorbance, benchmarked against the reference material, confirmed the successful formation of a neutral calcium sulfonate.

What is claimed is:

1. A method for producing sulfonates having a reduced carbon footprint using a re-refined base oil, comprising:
combining a re-refined base oil with a sulfonation agent to produce a sulfonated reaction mixture, wherein the sulfonation agent comprises SO₃;
obtaining an oil layer from the sulfonated reaction mixture;
neutralizing the oil layer and adding an inorganic base to the oil layer to produce a neutralized oil layer;
separating a sulfonate layer from the neutralized oil layer; and
isolating sulfonates from the sulfonate layer to produce sulfonates having a reduced carbon footprint.

2. The method of claim 1, wherein the re-refined base oil comprises less than 90% saturates and greater than 1% aromatic content.

3. The method of claim 1, wherein the sulfonation agent is combined with the re-refined base oil in an amount that provides SO₃ at about 3 to 5 weight percent of the re-refined base oil.

4. The method of claim 1, wherein the sulfonation agent is SO₃ gas, oleum, sulfuric acid, chloro sulfonic acid, acetyl sulfate, or combinations thereof.

5. The method of claim 1, wherein the step of neutralizing the oil layer further comprises adding isopropyl alcohol, water, methanol, or combinations thereof to the oil layer.

6. The method of claim 1, wherein the inorganic base comprises sodium, calcium, barium, lithium, magnesium, zinc, strontium, or ammonium.

7. The method of claim 1, further comprising reacting the sulfonates from the sulfonate layer with reagents to obtain over-based sulfonates having a reduced carbon footprint.

8. The over-based sulfonates having a reduced carbon footprint prepared by the method of claim 7.

9. The sulfonates having a reduced carbon footprint prepared by the method of claim 1.

10. A method for producing sulfonates having a reduced carbon footprint using a re-refined base oil, comprising:
combining a re-refined base oil with SO₃ gas or oleum to produce a sulfonated reaction mixture, wherein the re-refined base oil comprises less than 90% saturates and greater than 1% aromatic content, wherein the sulfonation agent comprises SO₃;
obtaining an oil layer from the sulfonated reaction mixture;
neutralizing the oil layer and adding Na₂CO₃ or Ca(OH)₂ to the oil layer to produce a neutralized oil layer;
separating a sulfonate layer from the neutralized oil layer; and
isolating sodium sulfonates or calcium sulfonates from the sulfonate layer to produce sodium sulfonates having a reduced carbon footprint or calcium sulfonates having a reduced carbon footprint.

11. The sodium sulfonates having a reduced carbon footprint or calcium sulfonates having a reduced carbon footprint prepared by the method of claim 10.

12. The method of claim 10, wherein the sulfonation agent is combined with the re-refined base oil in an amount that provides SO₃ at about 3 to 5 weight percent of the re-refined base oil.

13. The method of claim 10, further comprising reacting the sodium sulfonates or calcium sulfonates from the sulfonate layer with reagents to obtain over-based sodium sulfonates having a reduced carbon footprint or over-based calcium sulfonates having a reduced carbon footprint.

14. The over-based sodium sulfonates having a reduced carbon footprint or over-based calcium sulfonates having a reduced carbon footprint prepared by the method of claim 13.

15. A composition comprising sulfonates having a reduced carbon footprint, wherein the sulfonates are derived from re-refined base oil.

16. The composition of claim 15, wherein the sulfonates are metal sulfonates.

17. The composition of claim 15, wherein the sulfonates are over-based sulfonates.
